(19) United States
(12) Patent Application Publication
(10) Pub. No.: US 2021/0236640 A1
(43) Pub. Date: Aug. 5, 2021

(54) RAPID METHOD TO PREDICT STABILITIES OF PHARMACEUTICAL COMPOSITIONS CONTAINING PROTEIN THERAPEUTICS AND NON-REDUCING SUGARS

(71) Applicant: Merck Sharp & Dohme Corp., Rahway, NJ (US)

(72) Inventors: Arnab De, Edison, NJ (US); Chakravarthy Nachu Narasimhan, Scotch Plains, NJ (US)

(73) Assignee: Merck Sharp & Dohme Corp., Rahway, NJ (US)

(21) Appl. No.: 17/052,687

(22) PCT Filed: May 6, 2019
(86) PCT No.: PCT/US2019/030802

§ 371 Date: Nov. 3, 2020

Related U.S. Application Data
(60) Provisional application No. 62/670,219, filed on May 11, 2018.

(51) Int. Cl. A61K 47/26; A61K 9/08; A61K 47/18

(52) U.S. Cl. CPC A61K 47/26; A61K 47/183; A61K 9/08

(57) ABSTRACT

Provided is a method of determining the stability increase provided by a non-reducing sugar in a pharmaceutical composition containing a protein therapeutic, the method comprising: (i) providing: a first pharmaceutical composition comprising an aqueous solution of the protein therapeutic in the substantial absence of a non-reducing sugar, wherein the first pharmaceutical composition has a first B22 value and a second pharmaceutical composition comprising an aqueous solution of the protein therapeutic and the non-reducing sugar, wherein the second pharmaceutical composition has a second B22 value; (ii) determining the difference between the first and second B22 values; and (iii) predicting the stability increase provided by the non-reducing sugar based on the difference in B22 values.

### FIELD OF THE INVENTION

This invention relates to a rapid method of determining the stability increase provided by a non-reducing sugar in a pharmaceutical composition containing a protein therapeutic.

### BACKGROUND OF THE INVENTION

In developing pharmaceutical compositions containing antibodies, formulators seek to maintain an antibody's solubility, stability and potency of its antigen binding. Maintaining such properties is paramount in developing liquid compositions containing high concentrations of monoclonal antibodies (mAbs) which are typically associated with compositions administered by subcutaneously. However, there is a general consensus that development of high-concentration formulations of mAbs poses serious challenges with respect to the physical and chemical stability of the mAbs, such as increased formation of soluble as well as insoluble aggregates which enhance the probability of an immunogenic response as well as result in low bioactivity.

Including salts, surfactants, buffering agents, and stabilizers such as sugars in pharmaceutical composition can often address aggregation problems. For instance, clinically used pharmaceutical compositions of protein therapeutics often contain non-reducing sugars, e.g., sucrose, as stabilizing excipients. Formulation of antibody preparations requires careful selection of these excipients among others to avoid denaturation of the protein and loss of antigen-binding activity. Indeed the finding that one excipient stabilizes a liquid composition containing one protein therapeutic, does not necessarily mean that the same excipient may stabilize a composition containing a different therapeutic, due to the differences in the proteins' structures.

In addition, combinations of excipients are typically included in a pharmaceutical composition to alter different properties of the composition, such as its viscosity, surface tension, and pH, or to maintain the physical stability and bioactivity of the protein therapeutic. Currently available techniques may not be able to detect whether a particular excipient contributes to the overall stability of the composition when such techniques are performed on compositions containing a plurality of excipients.

Often, the determination of a composition's stability has been delayed until late in the development cycle of the protein therapeutic when greater quantities of the protein are available, and the composition's propensity to form aggregates can more reliably be determined. The ability to assess a composition's propensity for aggregation with smaller quantities of protein therapeutics could allow the optimization of pharmaceutical composition earlier in the drug development cycle, thereby avoiding further development expense. In addition, the ability to determine whether a pharmaceutical composition is physically stable with smaller quantities of protein, could allow formulators to more quickly select protein candidates which are appropriate for further development.

Formulators often turn to determining the diffusion interaction parameter (k_{D}) as a useful method for determining the stability of protein-containing compositions. A positive k_{D} (k_{D}>0) indicates repulsive protein-protein interaction, which has been observed to correlate with more stable protein-containing compositions, i.e., less protein aggregation. Formulators can rapidly measure k_{D} using dynamic light scattering, which can be performed on a sample in about 1 hour. The parameter is calculated from the concentration dependence of the measured diffusion coefficient of the sample, as indicated in the expression below, where Dₘ is the mutual (measured) diffusion coefficient, D₀ is the self-diffusion coefficient (the diffusion coefficient at zero concentration), and C is the sample concentration.

Dₘ=D₀(1+k_{D}C)

However, the present applicants have observed that changes in k_{D} are relatively insensitive to the addition of sucrose to the solution containing protein therapeutics. The addition of sucrose only results in a negligible change in k_{D}.

Another parameter that formulators assess when predicting the stability of protein-containing compositions is the composition's zeta potential. Zeta potential measures the magnitude of the electrostatic or charge repulsion/attraction between particles, and is one of the fundamental parameters known to affect stability. Its measurement provides detailed insight into the causes of dispersion, aggregation or flocculation, and can be applied to improve the preparation of stabilized formulation of dispersions, emulsions and suspensions. The zeta potential is calculated from Henry's equation using the Smoluchoski approximation:

μₑ=2εkₛζ3η

where μₑ is the electrophoretic mobility, ε is the dielectric constant or permittivity of the solution, kₛ is a model-based constant which from the Smoluchoski approximation is 1.5 and ζ is the zeta potential.

The zeta potential, or the “effective charge at the slipping or interaction plane” is considered to be one of the main drivers from the standpoint of colloidal stability. The greater the net charge, the greater the electrostatic repulsion between like particles. For antibodies and other proteins, the net charge is particularly important, due to the heterogeneity of the surface charge, which can lead to attractive dipole-dipole interactions at the higher concentrations typical of biotherapeutics. For antibodies exhibiting large dipole moments, the net charge must be large enough to counter these attractive interactions; otherwise, aggregation and increased viscosity at high sample concentration is probable.

Another technique employed to characterize protein-containing compositions and assess protein aggregation involves continuous quantitative monitoring of test compositions using static light scattering. For instance, the ARGEN platform from Fluence Analytics, New Orleans, La. USA, uses this technique. To determine colloidal stability, compositions are stored in a sample holder and stressed at a predetermined temperature for a defined period of time. The static light scattering signal is measured continuously through the time period. When the sample begins to aggregate, the light scattering signal increases. One of the ways to assess colloidal stability is to measure the ‘lag time’ which is the time taken for the light scattering signal to increase (or the time taken for the samples to aggregate). The greater the lag time for a given composition, the more stable is the composition.

This technique for determining the aggregation properties of protein-containing compositions typically requires heating of significant quantities of the therapeutic protein (e.g., approximately 50 mg of the therapeutic protein) at 40-50° C. for 2-10 hours to complete the analysis. Since the samples are heated, the protein samples cannot typically be recovered and used for assessing properties of the protein.

Accordingly, additional methods which rapidly predict the stability of pharmaceutical compositions containing protein therapeutics are desirable. In addition, identifying methods that can assess the stability of pharmaceutical compositions with minimal quantities of protein therapeutics is particularly desirable.

### SUMMARY OF THE INVENTION

In one embodiment (embodiment no. 1), the present invention provides a method of determining the stability increase provided by a non-reducing sugar in a pharmaceutical composition containing a protein therapeutic, the method comprising:

(i) providing:

a first pharmaceutical composition comprising an aqueous solution of the protein therapeutic in the substantial absence of a non-reducing sugar, wherein the first pharmaceutical composition has a first B₂₂ value and

a second pharmaceutical composition comprising an aqueous solution of the protein therapeutic and the non-reducing sugar, wherein the second pharmaceutical composition has a second B₂₂ value;

(ii) determining the difference between the first and second B₂₂ values; and

(iii) predicting the stability increase provided by the non-reducing sugar based on the difference in B₂₂ values.

### BRIEF DESCRIPTION OF THE DRAWINGS

FIG. 1 is a histogram showing the lag time (T_{agg}) as determined by an aggregation rate generator for five distinct formulations containing an IgG4 monoclonal antibody.

FIG. 2 is a histogram showing the diffusion interaction parameters (k_{D}) for five distinct formulations containing an IgG4 monoclonal antibody.

FIG. 3 is a histogram showing the zeta potentials (in mV) for five distinct formulations containing an IgG4 monoclonal antibody.

FIG. 4 is a histogram showing the second virial coefficients (B₂₂, in ×10⁵ mL/g²) as determined by static light scattering (SLS) for five distinct formulations containing an IgG4 monoclonal antibody.

FIG. 5 is a histogram showing the second virial coefficients (B₂₂, in ×10⁵ mL/g²) as determined by dynamic light scattering (DLS) for five distinct formulations containing an IgG4 monoclonal antibody.

FIG. 6 is a histogram showing the diffusion interaction parameters (k_{D}, in mL/g) for three distinct formulations containing an IgG1 monoclonal antibody.

FIG. 7 is a histogram showing the diffusion interaction parameters (k_{D}) for three distinct formulations containing an IgG1 monoclonal antibody.

FIG. 8 is a histogram showing the second virial coefficients (B₂₂, in ×10⁵ mL/g²) as determined by DLS for three distinct formulations containing an IgG1 monoclonal antibody.

FIG. 9 is a histogram showing the diffusion interaction parameters (k_{D}, in mL/g) for two distinct formulations containing an IgG1 monoclonal antibody.

FIG. 10 is a histogram showing the second virial coefficients (B₂₂, in ×10⁵ mL/g²) as determined by DLS for two distinct formulations containing an IgG monoclonal antibody.

FIG. 11 is a histogram showing the diffusion interaction parameters (k_{D}, in mL/g) for two distinct formulations containing an IgG1 monoclonal antibody.

FIG. 12 is a histogram showing the second virial coefficients (B₂₂, in ×10⁵ mL/g²) as determined by DLS for two distinct formulations containing an IgG monoclonal antibody.

FIG. 13 is a histogram showing the diffusion interaction parameters (k_{D}, in mL/g) for two distinct formulations containing an IgG1 monoclonal antibody.

FIG. 14 is a histogram showing the second virial coefficients (B₂₂, in ×10⁵ mL/g²) as determined by DLS for two distinct formulations containing an IgG monoclonal antibody.

### DETAILED DESCRIPTION OF THE INVENTION

The present invention provides a rapid method for directly and quantitatively comparing the stability of a protein therapeutic, when it is formulated in aqueous solutions with and without a stabilizing excipient, such as a non-reducing sugar, e.g., sucrose. The addition of sucrose only results in a negligible change in zeta potential. The method is capable of predicting the stability of protein therapeutic-containing compositions, even in the presence of other excipients such as salts, buffers and surfactants. The technique requires minimal quantities of the therapeutic protein, e.g., ≤0.5 mg to conduct the analysis. While not being bound by any specific theory, the present invention is based on the observation that the presently disclosed method of assessing protein-protein interactions predicts colloidal and thermal stability when the protein therapeutic is formulated in aqueous solutions containing a non-reducing sugar. In contrast to the observations provided by the present invention, the applicants have found that changes in zeta potential are virtually insensitive to the addition of sucrose to a solution containing protein therapeutics.

The method provided by the present invention involves determination of the osmotic second virial coefficient (B₂₂) to measure the stability of the protein therapeutic-containing composition. The more positive the value of B₂₂ is for a given sample composition in comparison to B₂₂ for a second composition, the more stable the composition.

Two methods can be used to calculate B₂₂. In the first, samples are prepared having different protein concentrations and the light scattering intensity from each sample is measured using static light scattering which measurement takes about 6 hours. The scattering intensities are referenced against a standard, such as toluene. The results are used to contruct a Debye plot using the Zimm equation.

KC/R_{Θ}=1/M_{W}P_{(Θ)}+2B₂₂C

In this equation, K is a constant, C is the sample concentration, R_{Θ} is the Rayleigh ratio (the ratio of scattered light to incident light), B₂₂ is the second virial coefficient, M_{W} is the sample molecular weight and P_{(Θ)} is the angular scattering dependence.

In the second method of calculating B₂₂, the coefficient is determined using a spectroscopic instrument such as a Zetasizer Instrument from Malvern Instruments Worldwide, Malvern, United Kingdom. To measure B₂₂, series dilutions of protein concentrations are prepared and loaded into a low-volume quartz batch cuvette and analyzed in dynamic light scattering (DLS) mode. The mean count rate from the DLS measurement is multiplied by the corrected attenuation factor calibrated by the standard (e.g., toluene), then converted to K*c/R and plotted against protein concentrations to obtain the Debye plot. B₂₂ is calculated by using the equation: K*c/R=1/M+2B₂₂*c, where K is the optical constant, c is protein concentration, R is the excess Raleigh ratio (measured by Malvern ZetaSizer), and M represents the molecular weight.

Normally, compositions having a high diffusion interaction parameter k_{D} also have a high B₂₂. Thus, excipients that increase k_{D} also increase B₂₂ since these two parameters are related by the formula:

k_{D}=2B₂₂M_{W}−(k_{f}+2ν)

where k_{f} is the sedimentation interaction parameter, ν is the partial specific volume, and M_{W} is the molecular weight. The applicants have surprisingly observed that only B₂₂, and not k_{D}, of a pharmaceutical composition increases upon addition of a non-reducing sugar (e.g., sucrose). Addition of a nonreducing sugar results in a negligible change in k_{D}. B₂₂ is the only parameter that is sensitive to the addition of nonreducing sugars to the composition. Thus, formulators should compare stability of the B₂₂ values of compositions to measure the increase in stability resulting from the addition of nonreducing sugar.

As noted above in the embodiment no. 1 in the Summary of the Invention, the present invention provides a method of determining the stability increase provided by a non-reducing sugar in a pharmaceutical composition containing a protein therapeutic.

In embodiment no. 2, the present invention provides the method as described in embodiment no. 1, wherein in step (ii) determining the difference between the first and second B₂₂ values comprises measuring the first and second B₂₂ values using static light scattering.

In embodiment no. 3, the present invention provides the method as described in embodiment no. 1, wherein in step (ii) determining the difference between the first and second B₂₂ values comprises measuring the first and second B₂₂ values using dynamic light scattering.

In embodiment no. 4, the present invention provides the method as described in any one of embodiment nos. 1, 2, or 3, wherein the first and second pharmaceutical compositions comprise at least one additional excipient which is a buffer, an isotonic agent (e.g., NaCl), or a surfactant.

In embodiment no. 5, the present invention provides the method as described in any one of embodiment nos. 1, 2, 3, or 4, wherein the non-reducing sugar is sucrose, trehalose, raffinose, or a combination thereof.

In embodiment no. 6, the present invention provides the method as described in any one of embodiment nos. 1, 2, 3, 4, or 5, wherein the protein therapeutic agent is an antibody or a combination of antibodies.

### Definitions and Abbreviations

The term “antibody” as referred to herein encompasses whole antibodies. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{HI}, C_{H2} and C_{H3}. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (for example, but not limited to, effector cells) and the first component (CIq) of the classical complement system. Antibodies may be derived from any mammal, including, but not limited to, humans, monkeys, pigs, horses, rabbits, dogs, cats, mice, etc. The term “antibody” refers to monoclonal antibodies, multispecific antibodies, human antibodies, humanized antibodies, camelised antibodies, chimeric antibodies, and anti-idiotypic (anti-Id) antibodies (including, for example, but not limited to, anti-Id antibodies to antibodies of the invention). Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

The terms “antibody derivatives” as referred to herein mean antigen binding fragments (i.e., “antigen-binding portions”) or single chains of antibodies Non-limiting examples of antibody derivatives include single-chain Fvs (scFv), single chain antibodies, single domain antibodies, Fab fragments, F(ab′) fragments, and disulfide-linked Fvs (sdFv).

The term, “buffer” or “buffering agent’ means an excipient which when present in a solution resists changes when an acid or alkali is added or when the solution is diluted. Exemplary buffers for use in the pharmaceutical formulations provided herein include, but are not limited, to histidine, citrate, phosphate, succinate, glycine, and acetate.

The terms “dynamic light scattering” (DLS), as will be recognized by those of skill in the art, is a technique that may be used to determine the size distribution profile of small particles in suspension or polymers in solution. In measuring DLS, temporal fluctuations are usually analyzed by means of the intensity or photon auto-correlation function (also known as photon correlation spectroscopy or quasi-elastic light scattering).

The terms “static light scattering” (SLS), as will be recognized by those of skill in the art, is a technique that measures the intensity of the scattered light to obtain the average molecular weight of a protein in solution. For light scattering analyses, a high-intensity monochromatic light is beamed in a solution containing the macromolecules, i.e., proteins. One or many detectors are used to measure the scattering intensity at one or many angles.

The term “excipient” means an inert substance which is commonly used as a diluent, vehicle, preservative, binder or stabilizing agent for drugs which imparts a beneficial physical property to a formulation, such as increased protein stability, increased protein solubility, and decreased viscosity. Examples of excipients include, but are not limited to, surfactants (for example, but not limited to, SDS, Tween 20, Tween 80, polysorbate, polysorbate 80 and nonionic surfactants), saccharides (for example, but not limited to, sucrose, trehalose, and raffinose), polyols (for example, but not limited to, mannitol and sorbitol), fatty acids and phospholipids (for example, but not limited to, alkyl sulfonates and caprylate). For additional information regarding excipients, see Remington's Pharmaceutical Sciences (by Joseph P. Remington, 18th ed., Mack Publishing Co., Easton, Pa.), which is incorporated herein in its entirety.

The term “nonreducing sugar” as used herein means a mono- or disaccharide sugar that cannot donate electrons to other molecules and therefore act cannot as a reducing agent. Examples of nonreducing sugars include sucrose, trehalose, and raffinose.

The term “protein therapeutic” means protein hormones, antibodies, nanobodies, Fc fusion proteins, anticoagulants, blood factors, bone morphogenetic proteins, engineered protein scaffolds, enzymes, growth factors, hormones, interferons, interleukins, and thrombolytics.

The terms “stability” and “stable” as used herein in the context of a liquid comprising a protein therapeutic (e.g., an antibody including antibody fragment thereof) refer to the resistance of the protein therapeutic in the formulation to aggregation, degradation or fragmentation under given manufacture, preparation, transportation and storage conditions. The “stable” compositions of the invention retain biological activity under given manufacture, preparation, transportation and storage conditions. The stability of the protein therapeutic can be assessed by degrees of aggregation, degradation or fragmentation, as measured by high performance size exclusion chromatography (HPSEC), static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference formulation. The overall stability of a comprising comprising a protein therapeutic can be assessed by various immunological assays including, for example, ELISA and radioimmunoassay using isolated antigen molecules.

The term “surfactant” as used herein means organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials. Examples of pharmaceutically acceptable surfactants include polysorbates (e.g., polysorbates 20 or 80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethylene glycol, polypropylene glycol, and copolymers of ethylene and propylene glycol (e.g., Pluronics, PF68 etc). Often surfactants are added to formulations to reduce aggregation.

The phrase “low to undetectable levels of aggregation” as used herein refers to samples containing no more than about 5%, no more than about 4%, no more than about 3%, no more than about 2%, no more than about 1% and no more than about 0.5% aggregation by weight of protein as measured by high performance size exclusion chromatography (HPSEC) or static light scattering (SLS) techniques.

The terms “substantial absence of a non-reducing sugar” mean, in the context of a pharmaceutical composition, that such composition contains an amount that does not contribute to the stabilization of the protein-therapeutic containing composition. For instance, in certain embodiments, the pharmaceutical compositions contain less than 1% (w/v) of non-reducing sugar (e.g., sucrose).

### Other Optional Components Present in Pharmaceutical Compositions:

In addition to the nonreducing sugar, the pharmaceutical compositions which are assessed by the methods of the present invention may also contain buffering agents, isotonic agents (e.g., salts) and surfactants.

The pharmaceutical compositions described herein suitably further comprise one or more buffers. The concentration of a buffer, in the pharmaceutical compositions described herein is generally in the range of about 10 mM to about 100 mM, more suitably about 15 mM to about 80 mM, about 15 mM to about 60 mM, about 20 mM to about 60 mM, about 20 mM to about 50 mM, about 20 mM to about 40 mM, about 20 mM to about 30 mi, or about 15 mM, about 20 mM, about 25 mM, about 30 mM, about 35 mM, about 40 mM, about 45 mM, about 50 mM, about 55 mM or about 60 mM, including any ranges or values within these ranges.

The pharmaceutical compositions described herein suitably further comprise an isotonic agent, such as a salt selected from the group consisting of: NaCl, KCl, CaCl₂), and MgCl₂. In a specific embodiment, pharmaceutical compositions of the invention comprise NaCl.

The pharmaceutical compositions described herein suitably further comprise a surfactant.

### EXAMPLES

The following examples are provided to more clearly describe the present invention and should not be construed to limit the scope of the invention.

Abbreviations employed herein include the following: B₂₂=osmotic second virial coefficient; g=gram; k_{d}=diffusion interaction parameter; Met=methionine; mg=milligram; mL=milliliters; mM=millimolar; mV=millivolt; mol=molar; PS80=polysorbate 80.

### Example 1: Comparing the Diffusion Interaction Parameter (k_{D}), Zeta Potential and Second Virial Coefficient (B₂₂) as Predictors for the Stability of an IgG4 Monoclonal Antibody Composition

Five formulations of mAb1 were prepared. mAb1 is an IgG4 anti-PD1 antibody. To prepare Formulation Nos. A1 and A2, 236.2 mg/mL of mAb1 was diluted to 50 mg/mL by addition of 10 mM histidine. The final pH of Formulation Nos. A1 and A2 were determined to be 5.3±0.3 and 6.3±0.3, respectively. To prepare Formulation Nos. A3 and A4, 236.2 mg/mL of mAb1 was diluted to 50 mg/mL by addition of 50 mM NaCl and 10 mM histidine. The final pH of Formulation Nos. A3 and A4 were measured to be 5.3±0.3 and 6.3±0.3, respectively. To prepare Formulation No. A5, 160 mg/mL of mAb1 in 10 mM histidine, 7% sucrose, 0.02% PS80 and 10 mM L-Met was diluted to 50 mg/mL using the corresponding placebo (10 mM histidine, +7% sucrose, 0.02% PS80 and 10 mM L-Met).

| Formulation | | |
|---|---|---|
| No. | | |
| (Colloidal) | Composition | Relative Stability |
| A1 | 50 mg/mL mAb1 (10 mM histidine, | +++ |
| | pH = 5.3 ± 0.3) | |
| A2 | 50 mg/mL mAb1 (10 mM histidine, | +++ |
| | pH = 6.3 ± 0.3) | |
| A3 | Formulation No. A1 + | + |
| | 50 mM NaCl | |
| A4 | Formulation No. A2 + | + |
| | 50 mM NaCl | |
| A5 | 50 mg/mL mAb1 (10 mM histidine, | +++++ |
| | pH = 5.8 ± 0.2) + 7% sucrose/0.02% | |
| | PS80/10 mM Met | |

Colloidal Stability (Aggregation Data)

The colloidal stabilities of Formulations Nos. A1-A5 were determined on an ARGEN (Aggregation Rate Generator) from Fluence Analytics, New Orleans, La. This instrument is a light scattering-based instrument that measures the pharmaceutical stability of therapeutic proteins. The instrument contained multiple (16) sample holders capable of precise control of thermal stressors. By continuously monitoring the state of its samples, ARGEN provided kinetic data yielding early detection of aggregation—and thus provided the rate of aggregation.

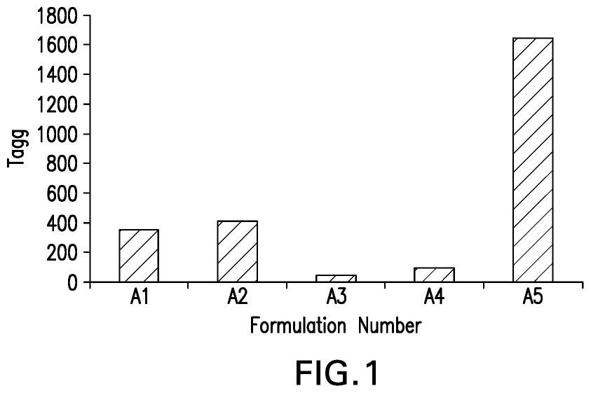

Formulation Nos. A1, A2, A3, A4 and A5 were stressed at 55° C. in the instrument. The “number of + units” in the Table above denotes the ‘relative stability’ as gleaned from FIG. 1, showing that A5 was the most stable, followed by A1 and A2—while A3 and A4 were the least stable. As shown in FIG. 1, data obtained using ARGEN shows that Formulation No. A5 (with sucrose) was the most stable formulation as it exhibited the longest lag-time (approximately 27 hours) before the onset of aggregation. In contrast, Formulation No. A1 had a lag-time of 6 hours, Formulation No. A2 had a lag time of 7 hours—while, Formulations Nos. A3 and A4 with NaCl were the least stable with lag-time of 1 hour and 2 hours respectively.

Diffusion Interaction Parameter (k_{D}) Determination

Various concentrations ranging from 20 mg/mL to 1 mg/mL were prepared from 50 mg/mL of mAb of the respective formulations. A Zetasizer Nano ZS instrument from Malvern Instruments (Malvern, United Kingdom) was used to determine the k_{D} values of each formulation. Briefly, about 100 μL of sample was taken in the ZEN2112 cell and the diffusion coefficients of the samples at various concentrations were measured at a temperature of 20° C.).

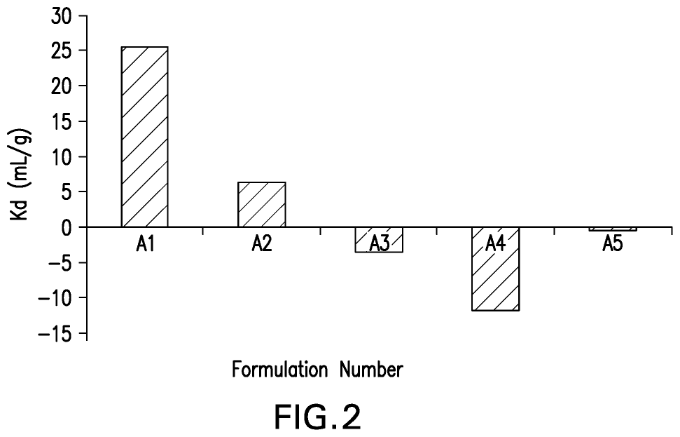

The k_{D} data shown in FIG. 2 correctly predicted that addition of NaCl destabilizes the formulation, i.e., Formulation Nos. A3 and A4 were less stable than Formulation Nos. A1 and A2. However, the k_{D} data failed to predict that Formulation No. A5 was the most stable formulation. Accordingly, this parameter proved insensitive to detecting the stability imparted by the addition of sucrose.

Zeta Potential Determination

The Zetasizer Nano ZS was also used to measure the electrophoretic mobility of the antibody via laser Doppler velocimetry and the zeta potential was calculated from Henry's equation using the Smoluchoski approximation. An antibody concentration of 10 mg/mL was used for all samples and the measurement was repeated on three samples at each condition and the errors are reported as the standard deviation. The temperature was controlled at 25° C.

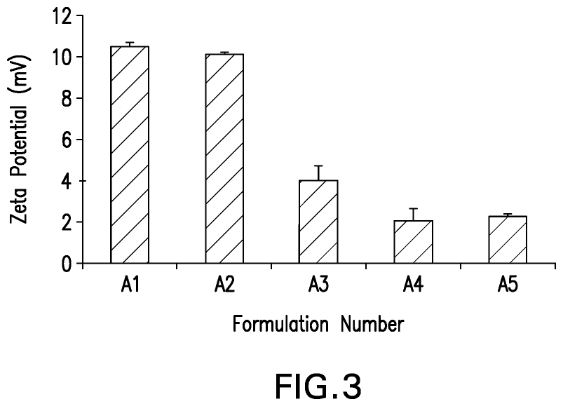

FIG. 3 shows the zeta potential of each formulation. The zeta potential data correctly predicted that addition of NaCl destabilized the formulation, i.e., Formulation Nos. A3 and A4 were less stable than Formulation Nos. A1 and A2 (which lacked NaCl). The zeta potential data reflected high conductivity in the presence of NaCl (as would be expected). However, like k_{D}, the zeta potential data also failed to predict that Formulation No. A5 was the most stable formulation. Accordingly, measurement of zeta potential proved insensitive to detecting the stability imparted by the addition of sucrose.

Second Virial Coefficient Determination

Two methods to determine the B₂₂ values of Formulation Nos. A1-A5. The first method used static light scattering measured on a Zetasizer APS instrument (Malvern, United Kingdom). The samples were prepared at different concentrations ranging from 15 mg/mL to 1 mg/mL at different concentrations, and the static light scattering intensity from each sample (at different concentration) was measured. The scattering intensities were referenced against standard (toluene). The results were used to build a Debye plot using the Zimm equation and B₂₂ was calculated.

The second method for determining B₂₂ used dynamic light scattering (DLS). Measurements were performed using the Malvern Zetasizer instrument. To measure B₂₂, series dilutions of protein concentrations were prepared, loaded into a low-volume quartz batch cuvette and analyzed in DLS mode. The mean count rate from the DLS measurement was multiplied by the corrected attenuation factor calibrated by toluene and the results were used to build a Debye plot using the Zimm equation and B₂₂ was calculated.

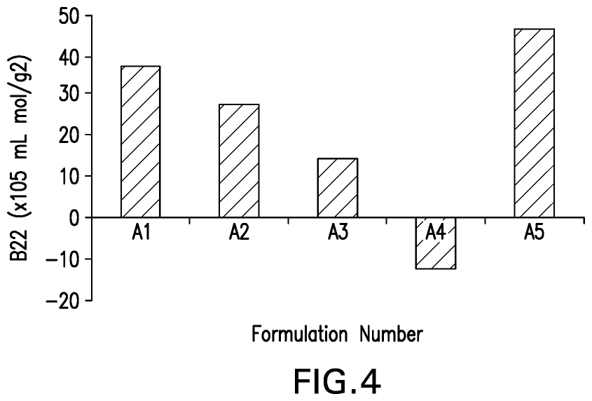

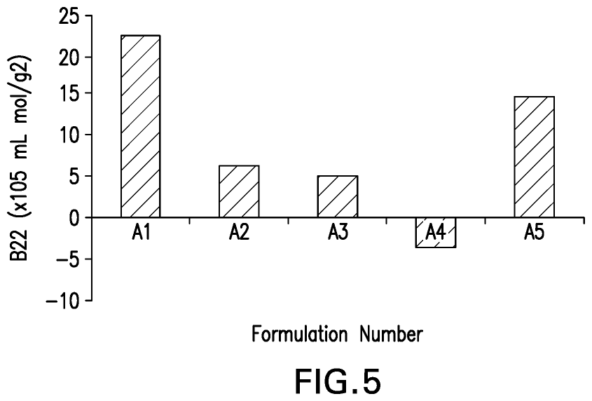

FIG. 4 shows the B₂₂ values of each formulation as determined by static light scattering. FIG. 5 shows the B₂₂ values of each formulation as determined by dynamic light scattering. The B₂₂ data (derived both from static and dynamic light scattering) correctly predicted that addition of NaCl destabilized the formulation, i.e., Formulation Nos. A3 and A4 were less stable than Formulation Nos. A1 and A2 (which lacked NaCl). Additionally, the B₂₂ data also predicted that Formulation No. A5 was be the most stable formulation. Accordingly, B₂₂ is a parameter that is sensitive to the addition of sucrose.

### Example 2: Comparing the Diffusion Interaction Parameter (k_{D}), Zeta Potential and Second Virial Coefficient (B₂₂) as Predictors for the Stability of an IgG1 Monoclonal Antibody Composition

Formulation Nos. B₁, B₂ and B₃ were prepared using stock solutions of 10 mM histidine (pH=6.0), 1 M NaCl and 40% sucrose. mAb2 is an anti-IL23 IgG1 antibody. As in Example 1, appropriate dilutions were made to yield the compositions described in the table below.

| Formulation No. | Composition |
|---|---|
| B1 | 50 mg/mL mAb2 (10 mM |
| | Histidine, pH = 6.0) |
| B2 | Formulation 1 + 7% Sucrose |
| B3 | Formulation 1 + 50 mM NaCl |

Diffusion Interaction Parameter (k_{D}) Determination

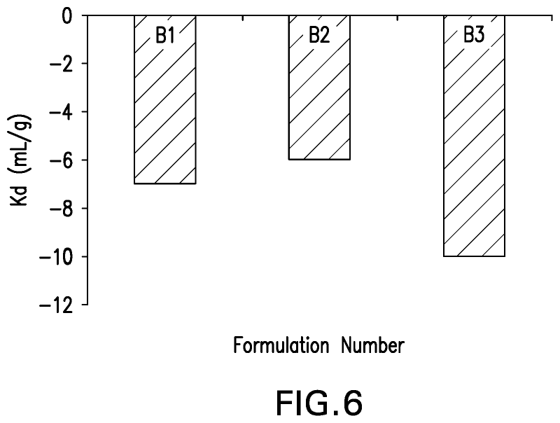

The k_{D} data for the formulations in Example 2 was generated using a similar method as described in Example 1. The k_{D} data shown in FIG. 6 predicts that addition of NaCl would destabilize the formulation as Formulation No. B₃ has a more negative k_{D} value as compared to Formulation No. B₁. The k_{D} data does not predict that Formulation No. B₂ would be the most stable formulation (i.e., the k_{D} value fails to predict a difference in stability caused by the addition of sucrose).

Zeta Potential Determination

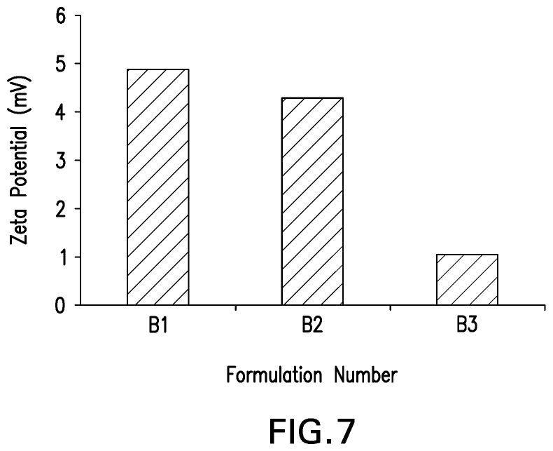

The zeta potential for formulations in Example 2 was generated using a similar method as described in Example 1. The zeta potential data as shown in FIG. 7 predicts that addition of NaCl would destabilize the formulation, i.e., Formulation No. B₃ would be less stable than Formulation No. B₁ (which lacked NaCl). It should be mentioned that the zeta potential data reflected high conductivity in the presence of NaCl (as would be expected). However, like k_{D}, the zeta potential data also does not predict that Formulation No. B₂ would be the most stable formulation.

Second Virial Coefficient Determination

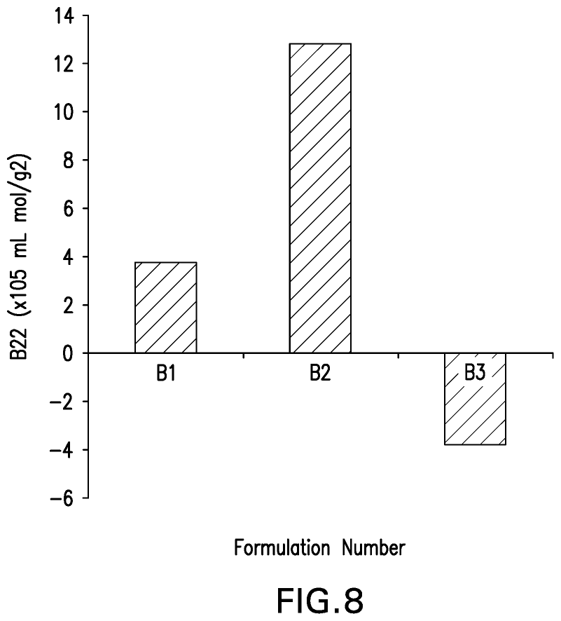

FIG. 8 shows the B₂₂ values of each formulation as determined by DLS. The B₂₂ values for formulations in Example 2 were generated using a similar method as described in Example 1.

The B₂₂ data for B₃ predicts that addition of NaCl would destabilize the formulation. Additionally, the B₂₂ data also predicts that B₂ would be the most stable formulation. B₂₂ is a parameter that is sensitive to the addition of sucrose.

### Example 3: Comparing the Diffusion Interaction Parameter (k_{D} and Second Virial Coefficient (B₂₂) as Predictors for the Stability of an IgG1 Monoclonal Antibody Composition

Formulation Nos. C1 and C2 contain mAb3 which is an anti-TIGIT (anti-T cell immunoglobulin and ITIM domain protein) IgG1 antibody. Formulation Nos. C1 and C2 were prepared by appropriate dilutions of relevant stock solutions):

Formulation No. C1—(50 mg/mL mAb3 in 10 mM histidine buffer, 10 m M L-Met, 7% sucrose, 0.02% polysorbate 80, pH=5.8)

Formulation No. C2—50 mg/mL mAb3 in 10 mM histidine buffer, pH 5.8.

| Formulation No. | Composition |
|---|---|
| C1 | 50 mg/mL mAb3 (10 mM |
| | histidine, pH = 5.8 ± 0.2) + 7% |
| | sucrose/0.02% PS80/10 mM Met |
| C2 | 50 mg/mL mAb3 (10 mM |
| | histidine, pH = 5.8) |

Diffusion Interaction Parameter (k_{D}) Determination

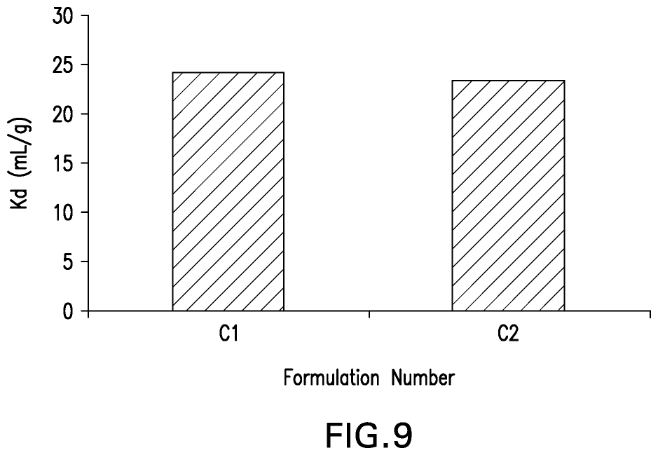

The k_{D} data for formulations in Example 3 was generated similar to the description in Example 1. As shown in FIG. 9, the k_{D} data does not predict differences in stability between the two formulations.

Second Virial Coefficient Determination

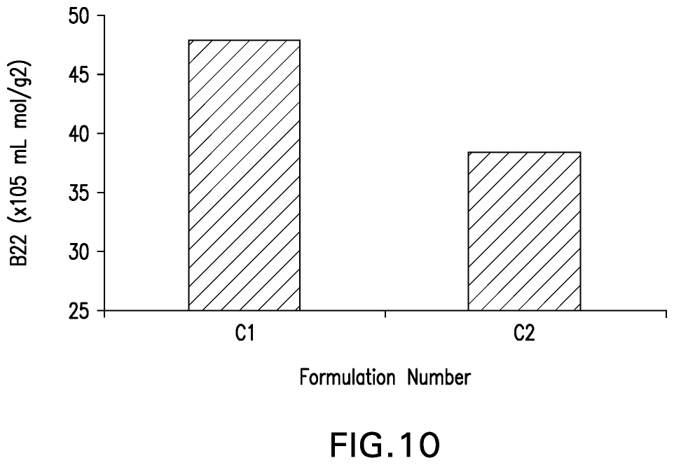

FIG. 10 shows the B₂₂ values of each formulation as determined by DLS. The B₂₂ values for formulations in Example 3 were generated similarly to the description provided in Example 1.

The B₂₂ data predicts that Formulation No. C1 (formulated with sucrose) would be the more stable formulation.

### Example 4: Comparing the Diffusion Interaction Parameter (k_{D} and Second Virial Coefficient (B₂₂) as Predictors for the Stability of an IgG1 Monoclonal Antibody Composition in the Presence of Sucrose in Acetate Buffer

Formulation Nos. D1 and D2 contained an IgG anti-CTLA4 (cytotoxic T-lymphocyte-associated protein 4) antibody, and were prepared by appropriate dilutions of relevant stock solutions).

| Formulation No. | Composition |
|---|---|
| D1 | 50 mg/mL mAb4 (10 mM |
| | Acetate, pH = 5.5) |
| | 50 mg/mL mAb4 (10 mM |
| D2 | Acetate, pH = 5.5 ± 0.2) + 7% |
| | sucrose/0.02% PS80/10 mM Met |

Diffusion Interaction Parameter (k_{D}) Determination

The k_{D} data for formulations in Example 4 was generated similarly to the description provided in Example 1.

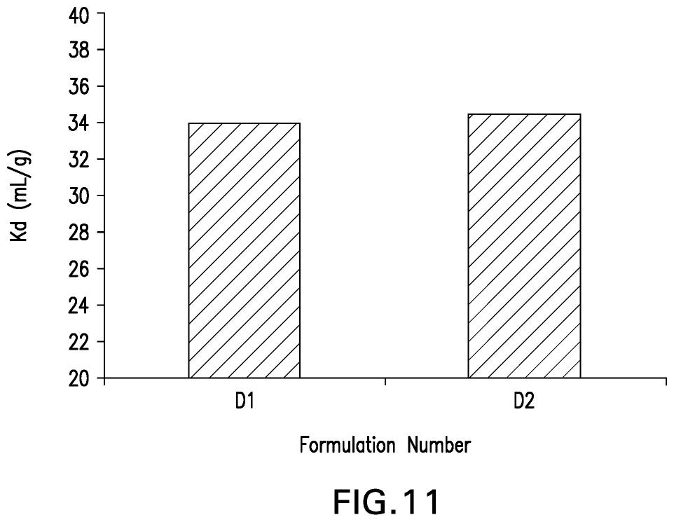

As shown in FIG. 11, the Kd data does not predict differences in stability between the two formulations.

Second Virial Coefficient Determination

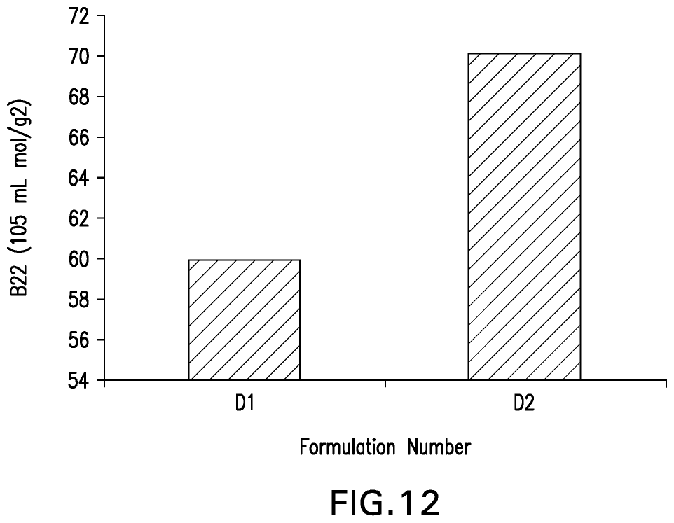

FIG. 12 shows the B₂₂ values of each formulation as determined by DLS. The B₂₂ values for formulations in Example 4 were generated similarly to the description provided in Example 1. The B₂₂ data predicts that Formulation No. D2 (formulated with sucrose) would be a more stable formulation than Formulation No. D1.

### Example 5: Comparing the Diffusion Interaction Parameter (k_{D} and Second Virial Coefficient (B₇₂) as Predictors for the Stability of an IgG1 Monoclonal Antibody Composition in the Presence of Sucrose in Histidine Buffer

Formulation Nos. E1 and E2 were prepared by appropriate dilutions of relevant stock solutions).

| Formulation Number | Composition |
|---|---|
| E1 | 50 mg/mL mAb4 (10 mM |
| | Histidine, pH = 5.5) |
| | 50 mg/mL mAb4 (10 mM |
| E2 | Histidine, pH = 5.5 ± 0.2) + 7% |
| | sucrose/0.02% PS80/10 mM Met |

Diffusion Interaction Parameter (k_{D}) Determination

The k_{D} data for formulations in Example 5 was generated similarly to the description provided in Example 1.

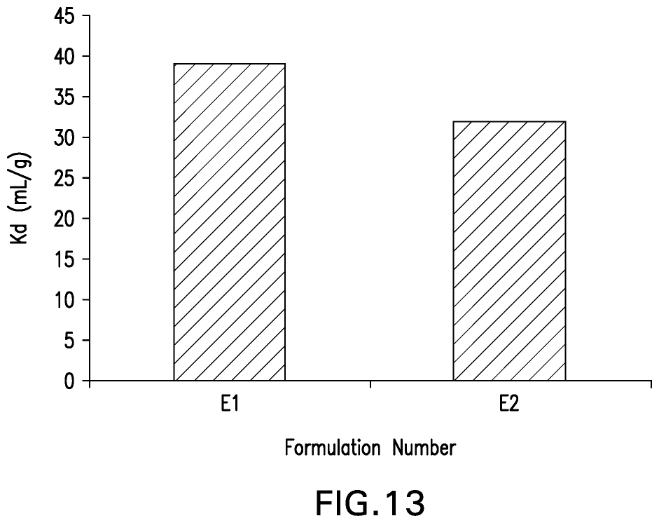

As shown in FIG. 13, the k_{D} data does not predict differences in stability between the two formulations.

Second Virial Coefficient Determination

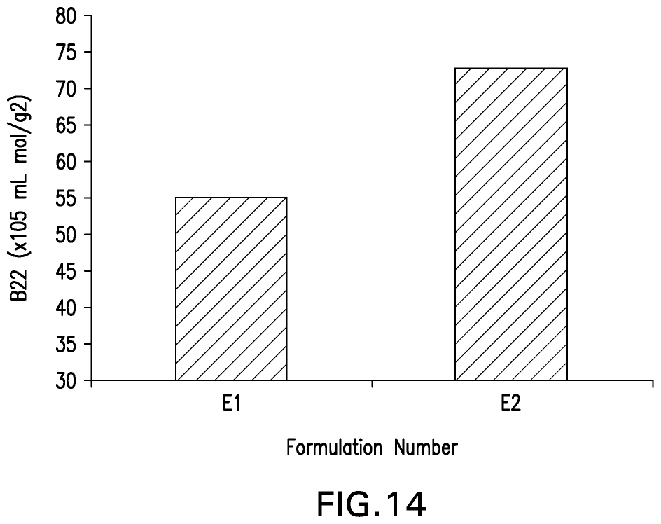

FIG. 14 shows the B₂₂ values of each formulation as determined by DLS. The B₂₂ values for formulations in Example 4 were generated similarly to Example 1.

The B₂₂ data correctly predicts that Formulation No. E2 (formulated with sucrose) would be a more stable formulation than Formulation No. E1.

What is claimed:

1. A method of determining the stability increase provided by a non-reducing sugar in a pharmaceutical composition containing a protein therapeutic, the method comprising:
(i) providing:
a first pharmaceutical composition comprising an aqueous solution of the protein therapeutic in the substantial absence of a non-reducing sugar, wherein the first pharmaceutical composition has a first B₂₂ value and
a second pharmaceutical composition comprising an aqueous solution of the protein therapeutic and the non-reducing sugar, wherein the second pharmaceutical composition has a second B₂₂ value;
(ii) determining the difference between the first and second B₂₂ values; and
(iii) predicting the stability increase provided by the non-reducing sugar based on the difference in B₂₂ values.

2. The method of claim 1, wherein, in step (ii) determining the difference between the first and second B₂₂ values comprises measuring the first and second B₂₂ values using static light scattering.

3. The method of claim 1, wherein, in step (ii) determining the difference between the first and second B₂₂ values comprises measuring the first and second B₂₂ values using dynamic light scattering.

4. The method of claim 1, wherein the first and second pharmaceutical compositions comprise at least one additional excipient which is a buffer, an isotonic agent or a surfactant.

5. The method of claim 4, wherein the additional excipient is the isotonic agent.

6. The method of claim 5, wherein the isotonic agent is NaCl.

7. The method of claim 1, wherein the non-reducing sugar is sucrose, trehalose, raffinose, or a combination thereof.

8. The method of claim 1, wherein the protein therapeutic agent is an antibody or a combination of antibodies.
